(19) United States
(12) Patent Application Publication
(10) Pub. No.: US 2017/0266053 A1
(43) Pub. Date: Sep. 21, 2017

(54) DISPOSABLE WARM COMPRESS EYE PATCH

(71) Applicant: Maria Rodriguez, Leesburg, VA (US)

(72) Inventor: Maria Rodriguez, Leesburg, VA (US)

(21) Appl. No.: 15/075,141

(22) Filed: Mar. 19, 2016

(51) Int. Cl. A61F 13/12; A61F 7/03; A61F 13/02; A61F 9/04; A61F 13/00

(52) U.S. Cl. CPC A61F 13/124; A61F 9/04; A61F 13/00063; A61F 13/00068; A61F 13/025; A61F 7/03; A61F 2013/0017; A61F 2013/00217; A61F 2007/0226

(57) ABSTRACT

The disposable eye patch further comprises a rim ember circumferentially attached to the second layer, wherein the rim member comprises an adhesive for sticking the eye patch to cover the human eye. The adhesive is a safe for contact with the human skin and can be easily removed and discarded after a single use. The disposable eye patch further comprises an absorbent layer attached to the first layer facing the outer surface of the eye.

### BACKGROUND

1. Field of the Invention

The present invention generally relates to devices for treating dry eye condition, and more specifically relates to a disposable warm compress eye patch with a self-adhering portion for sticking around the eye in order to provide comfort and pain relief to the ocular area.

2. Description of Related Art

Dry eye is a common condition in which, eyes are unable to produce sufficient amount of tears to lubricate and nourish the eyes. Glands such as Meibomian glands located in eyelids continually produce oil, which mixes with the tears in order to keep the eyes properly hydrated. Blockage of these glands causes reduction in production of tears and rapid evaporation of tears resulting in dry, gritty or irritated eyes. Blepharitis is another reason for eye discomfort, redness and tearing, burning and itching. Commonly adopted treatment options for dry eyes include the use of antibiotics, lubricants, frequent cleaning of eyelids and eye compresses.

Eye compresses generally employ cotton balls, soft woven cloth, liquid soaked gauze pads or gel filled masks for relieving swelling and providing comfort to the affected area around the eye. Some people even use warm potato, hot tea bags and hot towels as eye compress, which are generally not advisable by ophthalmologists. Conventional eye compresses in the form of eye mask or eye shield requires an adhesive band or a tape for attaching the eye mask to the eye region. Goggle type eye patches may shield the eye from dust, wind and light but does not provide adequate warm compress that is required for treating dry eye. Eyelid scrubs and detergent soaked pads are commonly used to alleviate problems associated with dry eyes.

Conventional eye patches are non-disposable and difficult to use, especially to hold over the surface of eyelid for a period. Frequent use of cotton wool, pad of flannel or soft fabric to clean eyelids and to provide warmth is cumbersome. Microwaveable gel packs or facemasks may cause burns due to excess amount of heat applied on the target site. Reusable eye pads or eye masks are difficult to clean and reuse. Moreover, reusable eye pads carry the risk of infection and does not constitute a hygienic option.

Therefore, there exists a need for a disposable eye patch for providing localized warm compress, which helps in relieving from dry eye and related conditions.

### SUMMARY OF THE INVENTION

The present invention relates to a disposable eye patch comprising a first layer circumferentially attached to a second layer. The first layer acts as an outer layer made of an impermeable material to prevent entry of extraneous substances such as dirt, dust and water. The second layer faces an external surface of eye when the eye patch is placed over the human eye. The two layers define a compartment there between filled with one or more heat generating material configured to produce an exothermic reaction for providing a warm compression to a human eye. The eye patch further comprises a rim member circumferentially attached to the second layer, wherein the rim member comprises an adhesive for removably sticking the eye patch to cover the human eye.

In an embodiment, the heat generating material may comprise one or more chemical substances, which generate heat upon exposing to air or other catalysts. The second layer presses against the outer surface of eye and made of a material to conduct heat. The first layer is made of an impermeable material to avoid heat loss and to render the layer water resistant. The eye patch may further comprise an additional absorbent layer attached to the second layer for absorbing discharge or fluid secreted from the eye. The rim member is made of a rubber material coated with adhesive substance.

Other features and advantages of the present invention will become apparent from the following more detailed description, taken in conjunction with the accompanying drawings which illustrate, by way of example, the principles of the invention.

### BRIEF DESCRIPTION OF DRAWINGS

FIG. 1A is a front view of the eye patch.

FIG. 1B is a sectional view of the eye patch of FIG. 1A broken along the line 1B-1B.

FIG. 2 is a rear view of the eye patch.

### DETAILED DESCRIPTION OF DIFFERENT EMBODIMENTS

The disclosure relates to an eye patch comprising a heat generating material for providing warm compress to eyes. The eye patch can be placed over the surface of eye and held in place with the help of a rim portion comprising an adhesive material. The eye patch is made of a disposable material, which allows the eye patch to be safely discarded after use.

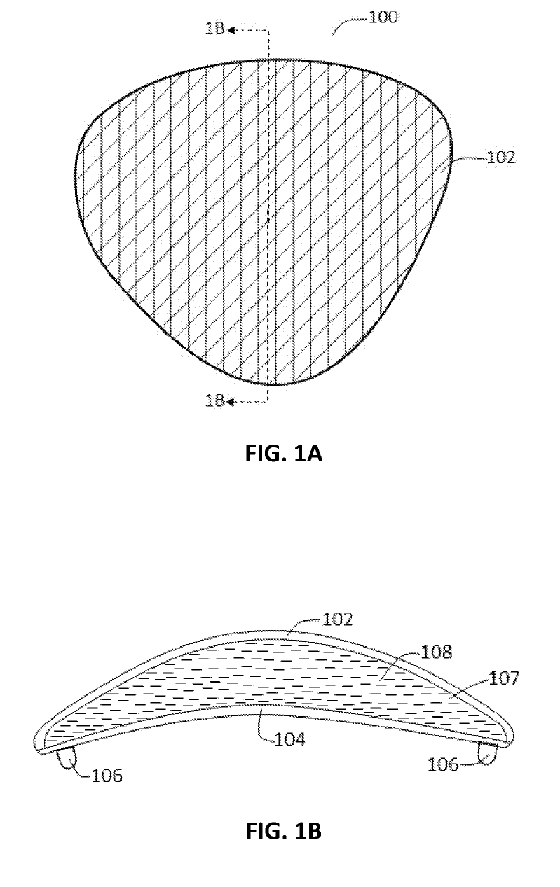

Referring to FIG. 1A, the front view of the eye patch 100 comprises an outer layer 102 made of an impermeable material in order to prevent entry of dust, air particulates, debris, water into the eye from the environment. The outer layer 102 is configured to fit to the contours of the eye and manufactured using an environment friendly, safely disposable material. In an embodiment, the outer layer 102 is made of a water resistant material.

FIG. 1B shows a sectional view of the eye patch 100 broken along the line 1B-1B. The outer layer 102 is attached along circumferential edges of an inner layer 104, thereby defining a space or compartment 108 in between the two layers 102 and 104. The compartment 108 is filled with a heat source or heat generating material 108 capable of producing an exothermic reaction upon exposure to air, water, other reactant or catalyst. The eye patch 100 further comprises a rim member 106 attached near the circumferential edge of the inner layer 104. The rim member 106 is made of a rubber material configured to stick the eye patch 100 over a human eye region. The inner layer 104 presses against the outer surface of eye and made of a material to conduct heat.

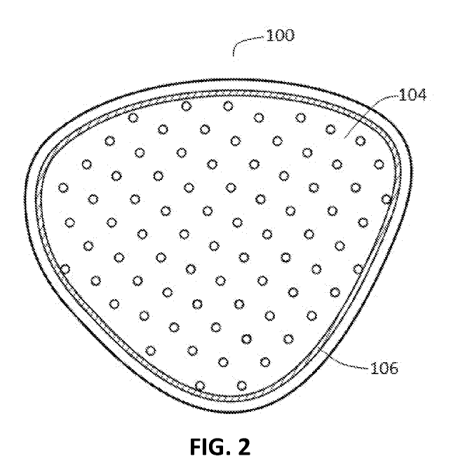

FIG. 2 is a rear view of the eye patch 100 showing the inner layer 104. The eye patch 100 comprises the rim member 106 attached adjacent to the circumferential edge of the inner layer 104. In an embodiment, the inner layer 104 comprises a self-adhesive rim member 106 that eliminates the need of tape, bandage, or straps for attaching and holding the eye patch over the eye surface. In an embodiment, the rim member 106 comprises a coating of an adhesive substance. In another embodiment, the self-adhesive rim member 106 may comprise a peel-off layer.

The eye patch 100 of the present invention is primarily used to providing warm compress to the eye especially during conditions such as dry eye, blepharitis, conjunctivitis, meibomitis, dacryocrystitis, chalazia, styes, orbital cellulitis, post-surgical recovery and the like. The heat generating material 108 may comprise one or more chemicals in a liquid or powdered form, capable of producing an exothermic reaction. These chemical substances may trigger the exothermic reaction upon exposure to air or oxygen, water or other chemical substances. In an embodiment, the heat generating material 108 within the eye patch 100 is capable of producing heat upon shaking or rubbing. The eye patch 100 acts as warm compress providing comfort and pain relief in the ocular area and facilitate in loosening of obstructions in pores or glands in the eye lids.

In an embodiment, the heat generating material 108 may comprise substances, which form an oxide when reacted with oxygen at room temperature, including iron, aluminium, magnesium, titanium, manganese, zinc, molybdenum and tin oxide (II). Further examples of suitable heat generating material 108 include metal sulphides, polysulphides or hydrosulphides mixed with a catalyst; Powdered iron, mixed with salts and water, mixtures of metal powder, water, cellulose, salt, activated charcoal, alkali metal salts and catalyst. It will be understood that some of the components of the mixtures listed do not directly contribute to the heat generating reaction but are present to modify or control the reaction. For example, catalysts, assistants, fillers and moisteners may be present.

Both temperature and duration of the heat are controlled to provide sufficient amount of heat without damaging tissue or skin around the eye. The duration of exothermic reaction can be controlled by the modifying the amount of chemical substance within the eye patch 100. In an embodiment, the eye patch 100 is configured to provide heat up to 30 minutes, preferably 15-20 minutes. The inner layer 104 is shaped according to the contours of eye region in order to provide a firm compress over the eye. The eye patch 100 may further comprise an absorbent layer attached to the inner layer 104, for absorbing discharge and secretions from the eye. In an embodiment, the absorbent layer may comprise a lubricant for eyes, antimicrobial solution or a cleansing agent.

Although an embodiment of the invention has been described in detail for purposes of illustration, various modifications may be made without departing from the scope and spirit of the invention. Accordingly, the invention is not to be limited, except as by the appended claims.

What is claimed is:

1. A disposable eye patch comprising:
a first layer circumferentially attached to a second layer defining a compartment there between filled with a least one heat generating material configured to produce an exothermic reaction for providing a warm compression to a human eye; and
a rim member circumferentially attached to the first layer, wherein the rim member comprises an adhesive for removably sticking the eye patch to cover the human eye.

2. The disposable eye patch of claim 1, further comprises an absorbent layer attached to the first layer.

3. The disposable eye patch of claim 1, wherein the rim member comprises a rubber material.

4. The disposable eye patch of claim 1, wherein the second layer is made of an impermeable material.

5. The disposable eye patch of claim 2, wherein the absorbent layer comprises a lubricant.

6. The disposable eye patch of claim 2, wherein the absorbent layer comprises an antimicrobial solution.

7. The disposable eye patch of claim 2, wherein the absorbent layer comprises a cleansing solution.

8. The disposable eye patch of claim 1, wherein the heat generating material comprises chemical substances, which generate heat when exposed to air.

9. The disposable eye patch of claim 1, wherein the heat generating material comprises chemical substances, which generate heat when exposed to water or catalyst.
